(19) United States
(12) Patent Application Publication
(10) Pub. No.: US 2009/0137553 A1
(43) Pub. Date: May 28, 2009

(54) Thiazepine Oxazolidinones as Antibacterial Agents

(75) Inventors: Charles Francis Donovan, Colchester, CT (US); Vara Prasad Venkata Nagendra Josyula, Superior Township, MI (US); Manjinder Singh Lall, East Lyme, CT (US); Adam Robert Renslo, Oakland, CA (US)

Correspondence Address: PFIZER INC;Steve T. Zelson, 150 EAST 42ND STREET, 5TH FLOOR - STOP 49, NEW YORK, NY, 10017-5612, US

(73) Assignees: Pharmacia and Upjohn Company LLC, Kalamazoo, MI (US); Pfizer Inc.

(21) Appl. No.: 11/577,997

(22) PCT Filed: Nov. 17, 2005
(86) PCT No.: PCT/IB2005/003552

§ 371 Date: May 22, 2008

Related U.S. Application Data
(60) Provisional application No. 60/631,476, filed on Nov. 29, 2004.

(51) Int. Cl. A61K 31/554; C07D 417/10; A61P 31/04

(52) U.S. Cl. 514/211.15; 540/544

(57) ABSTRACT

The present invention relates to a new class of oxazolidinone derivatives, to their use as antibacterial agents, to pharmaceutical compositions containing these compounds and to methods for their preparation.

### FIELD OF INVENTION

The present invention relates to a new class of oxazolidinone derivatives, to their use as antibacterial agents, to pharmaceutical compositions containing these compounds and to methods for their preparation.

### BACKGROUND OF THE INVENTION

Antibacterial resistance is a global clinical and public health problem that has emerged with alarming rapidity in recent years and undoubtedly will increase in the near future. Resistance is a problem in the community as well as in health care settings, where transmission of bacteria is greatly amplified. Because multiple drug resistance is a growing problem, physicians are now confronted with infections for which there is no effective therapy. As result, structurally novel antibacterials with a new mode of action have become increasingly important in the treatment of bacterial infections.

Among newer antibacterial agents, oxazolidinone compounds are the most recent synthetic class of antimicrobials. This invention provides a new class of oxazoldinone derivatives containing a thizaepine ring, which are active against a nunmber of human and veterinary pathogens, including multiple resistant strains of bacteria.

### Information Disclosure

WO 9323384, WO 20028084, WO 2003072553, WO 2003072576, WO 2003072575, WO 200142229, WO 200264575, WO 9615130, WO 200216960, WO 200027830, WO 200146185, WO 200281469, WO 200281470, WO 2001080841, WO 2003084534, WO 2003093247, WO 200202095, WO 200230395, WO 200272066, WO 2003063862, WO 2003072141, WO 2003072081, WO 2003119817, WO 2003008389, WO 2003007870, WO 200206278, WO 200032599, WO 9924428, WO 2004014392, WO 2004002967, WO 2004009587, WO 2004018439, US Patent Application Publication No. US 2004/0044052, U.S. Pat. No. 5,547,950, U.S. Pat. No. 5,700,799, DE 10034627 disclose oxazolidinone compounds having antibacterial activity useful for treating microbial infections.

### SUMMARY OF THE INVENTION

The present invention provides a compound of formula I

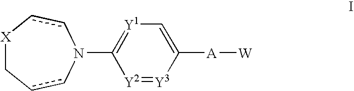

or a pharmaceutically acceptable salt thereof wherein:
A is a structure of the following formula i, ii, iii, or iv

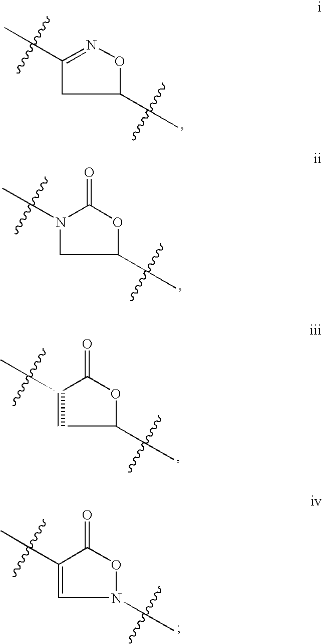

W is
   (a) CONHR¹,
   (b) CH₂NHR²,
   (c) CH₂OH,
   (d) CH(OH)—CH=CHR¹,
   (e) CH(OH)C≡CR¹,
   (f) CH₂NH-het,
   (g) CH₂O-het,
   (h) CH₂S-het, or
   (i) CH₂het;
X is S, SO, SO₂, or S=N—C(=O)C₁₋₆alkyl;
Y¹ is CH, CF, or N;
Y² and Y³ are independently CH or CF;
R¹ is H, C₁₋₆alkyl, or OC₁₋₆alkyl;
R² is C(=O)C₁₋₆alkyl, or CO₂(NH)C₁₋₆alkyl;
   each “. . . ” is independently a bond or absence;
   at each occurrence, C₁₋₆-alkyl is optionally substituted with one or more CF₃, halo, OH, OC₁₋₄alkyl, CN, N₃, O(C=O)C₁₋₄alkyl, C₃₋₆cycloalkyl, NH₂, NHC(=O)C₁₋₄alkyl, or C(=O)C₁₋₄alkyl;
het is a five- (5) or six- (6) membered heterocyclic ring having 1-4 heteroatoms selected from the group consisting of oxygen, sulfur, and nitrogen within the ring,
   wherein each carbon atom in het is optionally substituted with one or more CF₃, halo, OH, OC₁₋₄alkyl, CN, N₃, O(C=O)C₁₋₄alkyl, C₃₋₆cycloaLkyl, NH₂, NHC(=O)C₁₋₄alkyl, or C(=O)C₁₋₄alkyl; and with the proviso that where A is formula ii and each “. . . ” absence, then R² is other than C(=O)C₁₋₆alkyl.

In another aspect, the present invention also provides:

a pharmaceutical composition which comprises a pharmaceutically acceptable carrier and a compound of formula I,

a method for treating microbial infections in a mammal by administering to the subject in need a therapeutically effective amount of a compound of formula I or a pharmaceutically acceptable salt thereof, and

a use of a compound of formula I or a pharmaceutically acceptable salt thereof to prepare a medicament for treating microbial infections.

The invention may also provide novel intermediates and novel processes that are useful for preparing compounds of formula I.

### DETAILED DESCRIPTION OF THE INVENTION

Unless otherwise stated, the following terms used in the specification and claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆alkyl refers to alkyl of one to six carbon atoms, inclusive.

The term alkyl, or alkenyl, etc. refer to both straight and branched groups, but reference to an individual radical such as “propyl” embraces only the straight chain radical, a branched chain isomer such as “isopropyl” being specifically referred to.

The term “halo” refers to fluoro (F), chloro (Cl), bromo (Br), or iodo (I). The term “a pharmaceutically acceptable salt” of a compound means a salt that is pharmaceutically acceptable and that possesses the desired pharmacological activity of the parent compound.

The term “het” is a five- (5) or six- (6) membered heterocyclic ring having 1-4 heteroatoms selected from the group consisting of oxygen, sulfur, and nitrogen within the ring. An examples of het includes, but are not limited to, azetidine, pyrrole, imidazole, pyrazole, 1,2,3-triazole, 1,3,4-triazole, oxazole, thiazole, isoxazole, isothiazole, 1,3,4-oxadiazole, 1,3,4-thiadiazole, 1,2,3-thiadiazole, tetrazole, pyridine, pyrazine, pyrimidine, pyridazine, indolizine, isoindole, indole, dihydroindole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, iniidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazole, tetrazole, thiazolidine, thiophene, benzo[b]thiophene, morpholine, thiomorpholine, (also referred to as thiamorpholine,), piperidine, pyrrolidine, tetrahydrofuran, or the like. Another example of het includes, but are not limited to, pyridine, thiophene, furan, pyrazole, pyrimidine, 2-pyridyl, 3-pyridyl, 4-pyridyl, 2-pyliniidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-is-oxaz-olyl, 5-isoxaz-olyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,3-triazole-1-yl, 1,2,4-triazol-3-yl, 1,2,4-triazol-5-yl, tetrazole-1-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, or 1,2,4-dithiazolone.

The term “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human phamaceutical use. “A pharmaceutically acceptable carrier” as used in the specification and claims includes both one and more than one such carrier.

The term “mammal” refers to human or warm-blooded animals including livestock and companion animals. Livestock refers to animas suitable for human meat consumption. Examples include pigs, cattle, chickens, fish, turkeys, rabbits, etc. Companion animals refer to animals kept as pets such as dogs, cats, etc.

The term “optional” or “optionally” means that the subsequently described event or circumstance may, but need not, occur, and that the description includes instances where the event or circumstance occurs and instances in which it does not.

The term “treating” or “treatment” of a disease includes: (1) preventing the disease, i.e. causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “prodrug” refers to compounds that are rapidly transformed in vivo to yield the parent compound of the above formulas, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The term “leaving group” has the meaning conventionally associated with it in synthetic organic chemistry i.e., an atom or group capable of being displaced by a nucleophile and includes halogen, alkylsulfonyloxy, ester, or amino such as chloro, bromo, iodo, mesyloxy, tosyloxy, trifluorosulfonyloxy, methoxy, N,O-dimethylhydroxyl-amino, and the like.

Compounds that have the saine molecular formula but differ in the nature or sequence of bonding of their atoms or the arrangement of their atoms in space are termed “isomers”. Isomers that differ in the arrangement of their atoms in space are termed “stereoisomers”.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The compounds of the present invention are generally named according to the IUPAC or CAS nomenclature system.

Abbreviations which are well known to one of ordinary skill in the art may be used (e.g. “Ph” for phenyl, “Me” for methyl, “Et” for ethyl, “h” for an hour or hours and “rt” for room temperature).

Specific and preferred values listed below for radicals, substituents, and ranges, are for illustration only, they do not exclude other defined values or other values within defined ranges for the radicals and substituents.

Specifically, alkyl denotes both straight and branched groups; but reference to an individual radical such as “propyl” embraces only the straight chain radical, a branched chain isomer such as “isopropyl” being specifically referred to.

Specifically, alkyl is methyl, ethyl, propyl, isopropyl, butyl, iso-butyl, sec-butyl, and their isomeric forms thereof.

Specifically, alkenyl is vinyl, propenyl, allyl, butenyl, and their isomeric forms thereof.

Specifically, halo is fluoro (F), or chloro (Cl).

Specifically the present invention provides a compound of formula Ia

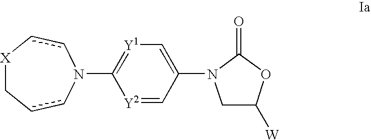

wherein Y² and Y³ are independently CH or CF; and W is CONHR², or CO₂(NH)C₁₋₆alkyl.

Specifically the present invention provides a compound of formula Ib

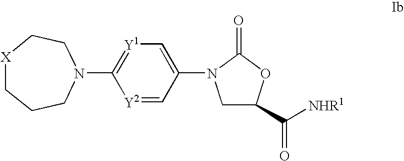

wherein Y² and Y³ are independently CH or CF.

Specifically, X is SO₂.

Specifically, R¹ is H or CH₃ or OCH₃.

Examples of the present invention are:
(1) 3-[3,5-difluoro-4-(1,4-thiazepan-4-yl)-phenyl]-2-oxo-oxazolidine-5-carboxylic acid amide,
(2) 3-[3,5-difluoro-4-(1,4-thiazepan-4-yl)-phenyl]-2-oxo-oxazolidine-5-carboxylic acid methylamide,
(3) 3-[4-(1,1-dioxo-1λ⁶-[1,4]thiazepan-4-yl)-3,5-difluoro-phenyl]-2-oxo-oxazolidine-5-carboxylic acid amide,
(4) 3-[4-(1,1-dioxo-1λ⁶-[1,4]thiazepan-4-yl)-3,5-difluoro-phenyl]-2-oxo-oxazolidine-5-carboxylic acid methylamide,
(5) 3-[4-(1,1-dioxo-1λ⁶-[1,4]thiazepan-4-yl)-3,5-difluoro-phenyl]-2-oxo-oxazolidine-5-carboxylic acid methoxy-amide,
(6) 3-{3,5-difluoro-4-[1-(2,2,2-triuoro-acetylimino)-114-[1,4]thiazepan-4-yl]-phenyl}-2-oxo-oxazolidine-5-carboxylic acid amide,
(7) 3-{3,5-difluoro-4-[1-(2,2,2-trifluoro-acetylimino)-114-[1 ,4]thiazepan-4-yl]-phenyl}-2-oxo-oxazolidine-5-carboxylic acid methylamide,
(8) (5R)-3-[3-fluoro-4-(1,4-thiazepan-4-yl)-phenyl]-2-oxo-oxazolidine-5-carboxylic acid amide,
(9) 5(R)-3-[3-fluoro-4-(1,4-thiazepan-4-yl)-phenyl]-2-oxo-oxazolidine-5-carboxylic acid methylamide,
(10) 5(R)-3-[4-(1-oxo-λ⁶-[1,4]thiazepan-4-yl)-3-fluoro-phenyl]-2-oxo-oxazolidine-5-carboxylic acid methyl ester,
(11) 5(R)-3-[-fluoro-4-(1-oxo-1λ⁴-[1,4]thiazepan-4yl)-phenyl]-2-oxo-oxazolidine-5-carboxylic acid methyl amide,
(12) 5(R)-3-[3-fluoro-4-(1-oxo-1λ⁴-[1,4]thiazepan-4-yl)-phenyl]-2-oxo-oxazolidine-5-carboxylic acid amide, or
(13) 5(R)-3-[4-(1,1-dioxo-1λ⁶-[1,4]thiazepan-4-yl)-3-fluoro-phenyl]-2-oxo-oxazolidine-5-carboxylic acid methyl amide.

Compounds of this invention can be prepared in accordance with one or more of the Schemes discussed below. All of the starting materials are either commercially available or can be prepared by procedures that would be well known to one of ordinary skill in organic chemistry. The variables used in the Schemes are as defined below, or as in the summary of the invention or claims.

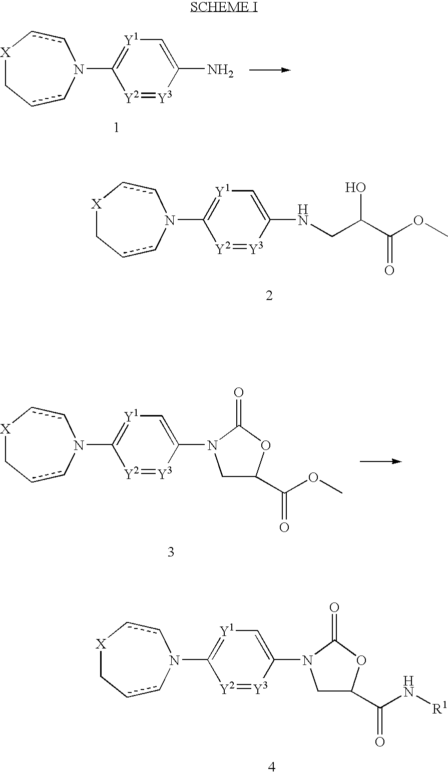

Scheme I describes the synthesis of the oxazolidinone ring with its C-5 carboxamide side chain, starting from commercially available aniline 1 (wherein X, Y¹, Y², Y³ and R² are as defined). First, the aniline 1 is reacted with an alkyl (2R)-epoxypropanoate and a Lewis acid such as ithium triflate (as described in US Patent Application Publication No. US 2004/0044052) to provide amino alcohol 2. Next the amino alcohol 2 is cyclized to give the aryl oxazolidinones 3 using methods known to one skilled in the art. For insance, treatment of intermediate 2 with 1,1′-carbonyldiimidazole in a solvent such as acetonitrile or tetrahydrofuran at an appropriate temperature, typically in a range of 20° C. to 80° C. provides the oxazolidinone 3. Alternatively, reaction of 2 with phosgene in a solvent such as toluene or methylene chloride, or mixtures thereof, im the presence of a base such as triethylamine at an appropriate temperature, typically in a range from −10° C. to 25° C., affords the oxazolidinone 3. The product may be used as collected or may first be purified using conventional techniques such as preparative TLC or HPLC, chromatography, precipitation, crystallization and the like.

Subsequent treatment of oxazolidinone ester 3 with ammonia or optionally with substituted amitnes (R²NH₂) in a suitable solvent such as methanol or acetonitrile affords amides 4 (R²=H or optionally substituted alkyl). Similarly, treatment of ester 3 with O-alkylhydroxylamines provides hydroxamates (R²=O-alkyl). The product may be used as collected or may first be purified using conventional techniques such as preparative TLC or HPLC, chromatography, precipitation, crystallization and the like.

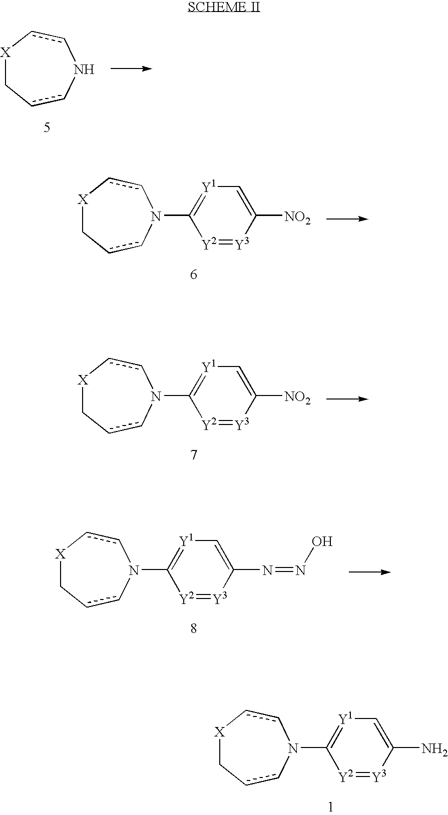

Scheme II describes the synthesis of aniline intermediates 1 bearing the thioazepanone ring. The thioazepanone 5 (for synthesis see Scheme III) is reacted in a nucleophllic aromatic substitution reaction with a fluoronitrobenzene (for example, with 3,4,-difluoronitrobenzene) to provide intermediates such as 6. Such reactions are well known those skilled in the art and review articles describing these reactions are available (see Zoltewicz in Top. Curr. Chem. 1975, vol. 59, pp. 33-64). These transformations are generally performed at a temperature in a range from about 40° C. to about 90° C. using polar aprotic solvents such as acetonitrile or dimethylformamide and in the presence of acid-scavenging bases such as triethylamine or N,N-diisopropylethylamine.

Intermediate 6 is then optionally alkylated on the nitrogen atom to form intermediate 7. For instance, reaction of 6 with methyl iodide using potassium hydroxide as a base and tetrabutylanmmonium bromide as a phase-transfer catalyst provides intermediate 7. Intermediate 7 is then optionally converted to intermediate 8 in which the amide has been converted to an amideoxime or similar functionality. These transformations are typically carried out in two steps, involving initial conversion of the amide to a thioamide followed by reaction of the thioamide with, for example, O-methyl hydroxylamine (to form an amideoxime). Finally, intermediate 8 is reduced to provide the aniline intermediate 1. This reduction is generally accomplished by reacting 8 with reducing metals (for example with iron powder). The reaction is favorably carried out at temperatures in a range from about 60° C. to about 90° C. in mixtures of water and alcohol (methanol, ethanol, etc.) as solvent, and in the presence of ammonium chloride to buffer the reaction mixture. Optionally, reductions of this type are conducted by reaction with other metals such as tin or zinc or by hydrogenation under palladium or platinum catalysis (see Rylander Hydrogenation Methods; Academic Press: New York, 1985, pp. 104-116). The product may be used as collected or may first be purified using conventional techniques such as preparative TLC or HPLC, chromatography, precipitation, crystallization and the like. Aniline intermediates such as 9 can then be converted to oxazolidinone analogs as described in Scheme I.

Schemes III-VI describe the synthesis of aryl isoxazolinone, aryl isoxazoline and aryl butyrolactone compounds bearing thiazepine and related heterocyclic rings of the type described in Schemes I-II. The following schemes describe general methods to prepare claimed structures in which A is (ii), (iii), or (iv). The starting materials required to prepare these compounds are aromatic aldehydes. The thiazepine and related heterocycles may be prepared as described above in Schemes I-II but it will be understood by those of ordinary skill in the art that suitable protecting groups may be required to protect and later reveal sensitive functional groups.

Scheme III summarizes the synthesis of the requisite substituted benzaldehyde intermediates. The general methods described in the previous Schemes are applicable but with the use of fluorinated benzonitrile (9) or benzoate ester starting materials in the place of fluorinated nitrobenzenes. The required starting materials (e.g., 3,4,5-trifluorobenzonitrile) are commercially available. Conversion of these starting materials to intermediate 10 is accomplished in a series of steps using the same methods described in Scheme I for the preparation of aniline intermediates. Intermediate 10 is then converted to the benzaldehyde intermediate 11 using methods that are well known to those of ordinary skill in the art, for example by reduction to the imine with SnCl2/HCl followed by hydrolysis (Stephen aldehyde synthesis).

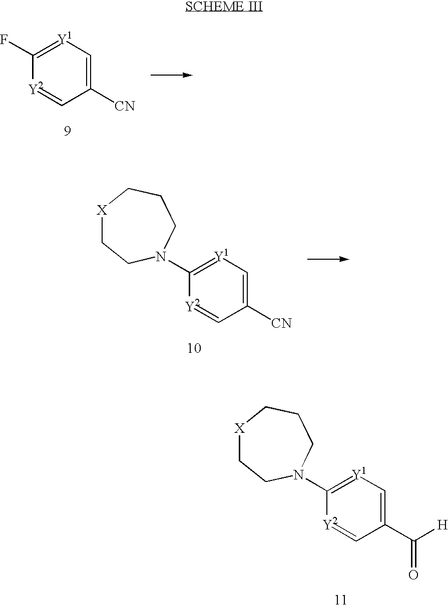

Scheme IV describes the preparation of aryl isoxazolinone analogs. The first step involves reaction of benzaldehyde intermediate 11 with ethyl diazoacetate (as described by Mahmood et al. in J. Org. Chem., 1998, 63, 3333-3336) to provide the ester aldehyde intermediate 12. Reaction of this material with hydroxylamine, followed by warming to reflux in aqueous methanol forms the isoxazolinone ring and intermediate 13. This intermediate is then converted to the corresponding methylacetamide 14 by reaction with N-(hydroxymethyl)acetamide acetate (prepared as described by Barnes et al. in U.S. Pat. No. 5,284,863) in a polar aprotic solvent such as DMF.

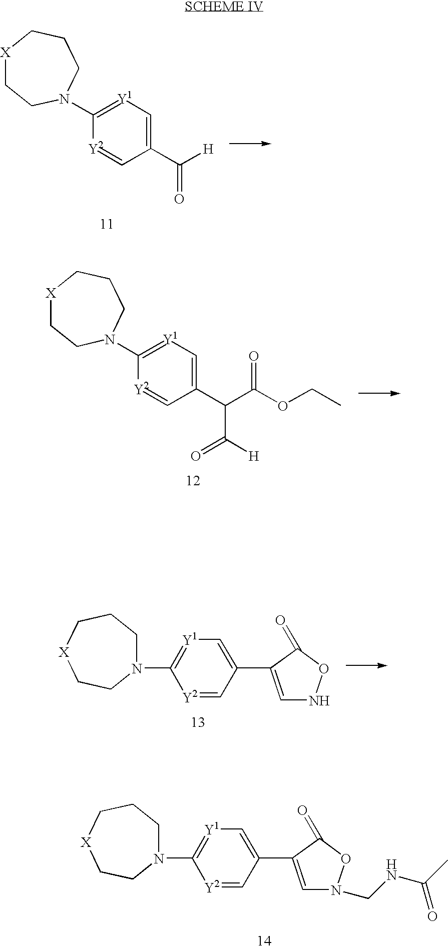

Scheme V describes a general method for preparing aryl isoxazoline compounds bearing diazepanone or related heterocycles of the type described in the Schemes above. In the first step of Scheme X the benzaldehyde intermediate 11 is reacted with hydroxylamine hydrochloride in a polar protic solvent, such as methanol, in the presence of a base, such as pyridine, to afford the oxime intermediate 15. The oxime 15 is then oxidized with N-chlorosuccinimide (NCS) in an appropriate solvent, such as dichloromethane, to give the hydroximinoyl chloride intermediate 16. This material is then reacted with an alkene, for example with allyl alcohol, in the presence of a base such as triethylamine and in a solvent such as dichloromethane, to provide the hydroxymethyl-substituted isoxazoline 17. The hydroxymethyl function of 17 may then be converted to acetamidomethyl or related moieties (e.g. 18, where R′=Et, OMe, etc.) using established synthetic procedures (for example, as described in US PCT publication US2004/0127530) to provide 18. Alternatively, the hydroximinoyl chloride 16 may be reacted with N-acetylallylamine to directly provide acetamidomethyl-substituted isoxazoline products (18, R′=Me). Optionally, the hydroximinoyl chloride intermediate may be formed in situ as described above and then directly treated with the alkenes to generate the isoxazoline intermediates directly from 15.

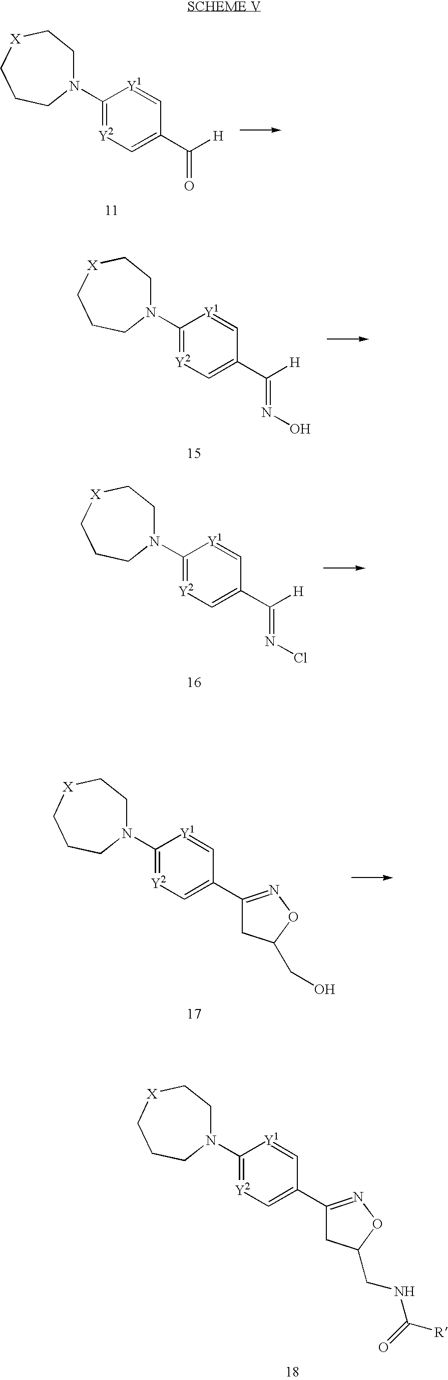

Scheme VI describes the synthesis of aryl butyrolactone analogs such as 22. The synthesis of the saturated (as in 21) and unsaturated (as in 22) 3-arylbutyrolactone ring system is described in the literature (for example, see Bioorganic & Medicinal Chemistry Letters, 1994, 4, 1925-1930). Aldehyde intermediate 11 is converted to phenyl acetic acid intermediate 39 using established procedures (for example as described by Hester, et al. in U.S. Pat. No. 5,708,169). The dianion of 19 is then reacted with R-benzyloxymethyloxirane in THF. The resulting hydroxyacid is cyclized under acid catalysis (for example, using p-toluenesulfonic acid) to provide lactone 20. The benzyl group is then removed by hydrogenolysis and the resulting hydroxymethyl function converted to acetamidomethyl or related moieties (e.g. 21, where R′=Et, OMe, etc.) using established synthetic procedures (for example, as described in US PCT publication US2004/0127530) to provide 21. Finally the butyrolactone is oxidized using a two-step protocol involving bromination (using for example N-bromosuccinimide) and subsequent elnnination by treatment with a suitable base such as pyridine or DBU to provide 22.

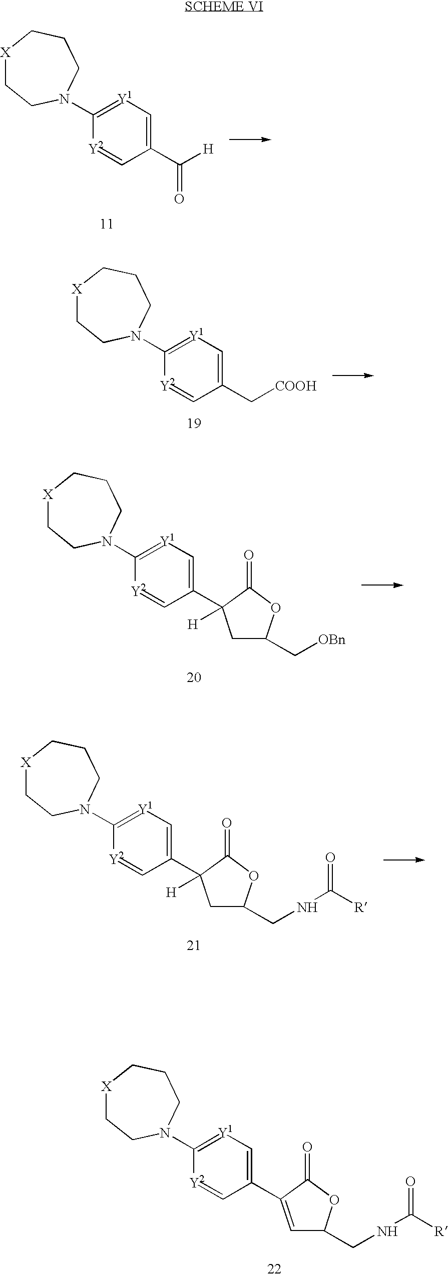

### Medical and Veterinary Uses

The compounds of the present invention may be used for the treatment of infectious diseases caused by a variety of bacterial organisms.

Examples include gram-positive bacteria such as multiple resistant staphylococci, for example S. aureus and S. epidermidis; multiple resistant streptococci, for example S. pneumoniae and S. pyogenes; and multiple resistant Enterococci, for example E. faecalis; gram negative aerobic bacteria such as Haemophilus, for example H. influenzae and Moraxella, for example M. catarrhalis; as well as anaerobic organisms such as bacteroides and clostridia species, and acid-fast organisms such as Mycobacteria, for example M. tuberculosis; and/or Mycobacterium avium. Other examples include Escherichia, for example E. coli. intercellular microbes, for example Chlamydia and Rickettsiae.

Examples of infections that may be treated with the compounds of the present invention include central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicernia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. Specifically, infectious diseases that may be treated with the compounds of the present invention are gram-positive infections such as osteomyelitis, endocarditis and diabetic foot.

### Antibacterial Activity

The in vitro antibacterial activity of the compounds of the present invention may be assessed by following procedures recommended in (1) National Committee for Clinical Laboratory Standards (January 2003), Methods for dilution antimicrobial tests for bacteria that grow aerobically, Approved Standard (6^{th} ed), M7-A6, NCCLS, Wayne, Pa.; (2) National Committee for Clinical Laboratory Standards (March 2001), Methods for antimicrobial susceptibility testing of anaerobic bacteria, Approved Standard (5^{th} ed), M11-A4, NCCLS, Wayne, Pa.; (3) National Committee for Clinical Laboratory Standards (January 2003), MIC testing supplemental tables, M100-S13 (for use with M7-A6), NCCLS, Wayne, Pa.; and (4) Murray P R, Baron E J, Jorgensen J H, et al. Manual of Clinical Microbiology (8^{th} ed) Washington, D.C.: American Society for Microbiology Press, 2003. The antibacterial activity can be presented in the form of MIC value. The MIC value is the lowest concentration of drug which prevented macroscopically visible growth under the conditions of the test.

TABLE 1
(Minimum Inhibitory concentrations μg/mL)

| | S. aureus | S. pyogenes | S. pneumoniae |
|---|---|---|---|
| Example | (UC-76) | (C203) | (SV-1) |
| 1 | 2 | 2 | 4 |
| 2 | 2 | 2 | 4 |
| 3 | 2 | 2 | 4 |
| 4 | 2 | 2 | 4 |
| 5 | 4 | 4 | 8 |
| 6 | 8 | 8 | 8 |
| 7 | 8 | 8 | 8 |
| 9 | 8 | 4 | 8 |
| 11 | 2 | 1 | 4 |
| 12 | 2 | 1 | 4 |
| 13 | 12 | 1 | 2 |

### Pharmaceutical Salts

The compound of formula I may be used in its native form or as a salt. In cases where forming a stable nontoxic acid or base salt is desired, administration of the compound as a pharmaceutically acceptable salt may be appropriate. Examples of pharmaceutically acceptable salts of the present invention include inorganic salts such as hydrochloride, hydrobromide, sulfate, nitrate, bicarbonate, carbonate salts, and organic salts such as tosylate, methanesulfonate, acetate, citrate, rnalonate, tartarate, succinate, benzoate, ascorbate, etoglutarate, and glycerophosphate.

Pharmaceutically acceptable salts may be obtained using standard procedures well known in the art, for example, reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Routes of Administration

In therapeutic use for treating, or combating, bacterial infections in a mammal (i.e. human and animals) a compound of the present invention or its pharmaceutical compositions can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

### Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art.

Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also fluction as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

The compounds may also be formulated for parenteral administration, e.g., by injections, bolus injection or continuous infusion. Formulations for parenteral admi tration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

For suppository admilustration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, compounds of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an hihaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more phamarceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compounds may also be formulated as depot preparations. Such long acting formulations may be in the form of inplants. A compound of this invention may be formulated for this route of administration with suitable polymers, hydrophobic materials, or as a sparing soluble derivative such as, without limitation, a sparingly soluble salt.

Additionally, the compounds may be delivered using a sustained-release system. Various sustained-release materials have been established and are well known by those skilled in the art. Sustained-release capsules may, depending on their chemical nature, release the compounds for 24 hours or for up to several days.

### Dosage

Pharmaceutical compositions suitable for use in the present invention include compositions wherein the active ingredients are contained in an amount sufficient to achieve the intended purpose, i.e., the treatment or prevent of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, a therapeutically effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the bacterial infection being treated. In average, the effective amount of active component is about 200 mg to 800 mg and preferable 600 mg per day.

The desired dose may conveniently be presented in a single dose or as divided doses administered at appropriate intervals, for example, as two, three, four or more sub-doses per day. The sub-dose itself may be flurther divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

### Oral Efficacy

### EXAMPLES

In the discussion above and in the examples below, the following abbreviations have the following meanings. If an abbreviation is not defined, it has its generally accepted meaning.
bm=broad multiplet
BOC=tert-butoxycarbonyl
bd=broad doublet
bs=broad singlet
bt=broad triplet
CDI=1,1O-carbodiimidazole
d=doublet
dd=doublet of doublets
dq=doublet of quartets
dt=doublet of triplets
dm=doublet of multiplets
DMF=dimethylfornamide
DMAP=dimethylamninopyridine
DIEA=diisopropylethylamine
DMSO=dimethyl sulfoxide
eq. equivalents
g=grams
h=hours
HPLC=high pressure liquid chromatography
HATU=N-[(dimethylamino)-1H-1,2,3-triazolo-[4,5-b]pyridin-1-yl-methylene]-N-methylmethanaminium hexafluorophosphate N-oxide
LG=leaving group
m=multiplet
M=molar
M %=mole percent
max=maximum
meq=milliequivalent
mg=milligram
mL=milliliter
mm=millimeter
mmol=millimol
q=quartet
s=singlet
t or tr=triplet
TBS=tnibutylsilyl
TFA=tifluoroacetic acid
THF=tetrahydrofuran
TLC=thin layer chromatography
p-TLC=preparative thin layer chromatography
μL=microliter
N=normality
MeOH=methanol
DCM=dichloromethane
HCl=hydrochloric acid
ACN=acetonitrile
MS=mass spectrometry
rt=room temperature
EtOAc=ethyl acetate
EtO=ethoxy
Ac=acetate
NMP=1-methyl-2-pyrrolidinone
μL=microliter
J=coupling constant
NMR=Nuclear magnetic resonance
MHz=megahertz
Hz=hertz
m/z=mass to charge ratio
min=minutes
Boc=tert-butoxycarbonyl
CBZ=benzyloxycarbonyl
DCC=1,3-dicyclohexylcarbodiimide
PyBop benzotriazole-1-yl-oxy-trispyrrolidinophosphonium hexafluorophosphate

### Example 1

### Preparation of 3-[3,5-difluoro-4-(1,4-thiazepan-4-yl)-phenyl]-2-oxo-oxazolidine-5-carboxylic acid amide

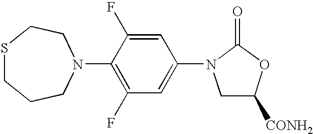

A 5 mL flask charged with 3-[3,5-difluoro-4-(1,4-thiazepanyl)-phenyl]-2-oxo-oxazolidine-5-carboxylic acid methyl ester 7 (0.1 g, 0.27 mmol) is treated with a methanolic solution of ammonia (1.3 mL, 2M in MeOH, 2.7 mmol) at room temperature with stirring for 17 h. The solvent is removed under reduced pressure and the crude material is dissolved in DCM/MeOH (10:1) and purified by preparative chromatograph (SiO₂, DCM-MeOH 5%), to afford the title compound as a yellow solid.

¹H NMR (400 MHz, CDCl₃) δ 7.10 (m, 2H), 6.57 (s, 11H), 5.70 (s, 11H), 4.99 (dd, 1H), 4.21 (m, 2H), 3.49 (m, 4H), 2.92 (dd, 2H), 2.82 (dd, 2H), 1.90 (m, 2H); MS-APCI (m/z+) 358 (M+H).

Intermediates for the synthesis of Example 1 are prepared as follows:
I. Preparation of 1,4-thiazepan-5-one

(Ref: J. Org. Chem. 1960, 25, 1953-1956.) To a stirred solution of tetrahydrothiopyran-4-one 1 (10 g, 86 mmol) in conc. HCl (43 mL) cooled to 0° C. is added sodium azide (8.4 g, 129 mmol) portionwise over 30-60 min, (note: gas evolution). When the addition is complete the reaction mixture is stirred at RT for 4 h.

The mixture is cooled to 0° C. and solid Na₂CO₃ is added portionwise (note: gas evolution), plus water to dissolve the salts, until the solution is alkaline ˜pH 9. The alkaline solution is diluted with DCM (200 mL), the phases are seperated and the aqueous layer is extracted with DCM (3×100 mL). The combined organic layers are dried over MgSO₄, filtered and concentrated to a low volume (˜20 mL), petroleum ether is added and the solid is collected by filtration and dried to afford the title compound as a white solid: ¹H NMR (400 MHz, DMSO) δ 7.52 (s, 1H), 3.39 (m, 2H), 2.63 (m, 2H), 2.58 (m, 4H); MS-APCI (m/z+) 131.9 (M+H).

II. Preparation of 1,4-thiazepane

(Ref: J. Org. Chem. 1960, 25, 1953-1956.) To a stirred solution of 1,4-thiazepan-5-one (9.16 g, 70 mmol) in THF (271 mL) at 0° C. is added dropwise over 20 min LAH (1M in THF, 70 mL, 70 mol). The reaction mixture is stirred at 0° C. for 10 min and then warmed to RT, stirring is continued for 2 h. The reaction mixture is quenched with careful successive addition of H₂O (2.5 mL), 5N aqeous NaOH (2.5 mL) and H₂O (9 mL). A thick gel like precipitate formed. The reaction mixture is filtered through a small pad of Celite, and the filter cake is washed with ether (300 mL). The filtrate is concentrated to afford the title compound as an oil, which is used immediately in the next reaction.

¹H NMR (400 MHz, DMSO) δ 2.89 (t, 2H), 2.84 (t, 2H), 2.69 (t, 2H), 2.59 (t, 2H), 1.78 (m, 2H).

III. Preparation of 4-(2,6-difluoro-4-nitro-phenyl)-1,4-thiazepane

A solution of 1,2,3-trifluoro-5-nitro-benzene (11.2 g, 63 mmol) in anhydrous acetonitrile (90 mL) is treated with triethylamine (9.7 mL, 70 mmol), followed by slow addition of 1,4-thiazepane (8.2 g, 70 mmol) suspended in acetonitrile (11 mL) over 30 min. The mixture is stirred at 23° C. for 15 h, then quenched by the addition of H₂O (100 mL) producing a yellow precipitate. The solution is filtered and the yellow solid is collected and dried in vacuo to give the title compound.

¹H NMR (400 MHz, DMSO) δ 7.90 (m, 2H), 3.63 (m, 4H), 2.81 (t, 2H), 2.61 (2H), 1.82 (m, 2H); MS-APCI (m/z+) 274.9 (M+H).

IV. Preparation of 3,5-difluoro-4-(1,4-thiazepanyl)-phenylamine

A solution of 4-(2,6-difluoro4-nitro-phenyl)-1,4-thiazepane (8.4 g, 31 mmol) in absolute ethanol (50 mL) is treated with SnCl₂2H₂O (28 g, 122 mmol) and refluxed for 1.5 h. The mixture is allowed to cool to room temperature and ice water is added, followed by the addition of 50% aqueous NaOH to pH 11. The solution is stirred for 1 h at room temperature and then extracted with CH₂Cl₂ (3×100 mL). The combined organic layers are washed with brine (300 mL), dried over MgSO4, and the solvent is removed in vacuo The crude title compound is used without further purification.

¹H NMR (400 MHz, DMSO) δ 6.17 (m, 2H), 5.37 (s, 2H), 3.18 (m, 4H), 2.79 (t, 2H), 2.60 (t, 2H), 1.80 (m, 2H); MS-APCI (m/z+) 245 (M+H).

V. Preparation of 3-[3,5-difluoro-4-(1,4-thiazepany)-phenylamino]-2-hydroxypropionic acid methyl ester.

A solution of 3,5-difluoro-4-(1,4-thiazepanyl)-phenylamine (7.9 g, 32 mmol) in anhydrous acetonitrile (15 mL) is treated with LiOTf (5 g, 32 mmol) and (R)-methyl glycidate (3.3 g, 32 mmol) and refluxed for 15 h. After 15 h, the reaction is incomplete therefore additional (R)-methyl glycidate (3.3 g, 32 mmol) is added and the solution is refluxed for another 15 h. The mixture is allowed to cool to room temperature and quenched by the addition of H₂O (20 mL), then extracted with EtOAc (3×50 mL). The combined organic layers are washed with brine (100 mL), dried over MgSO4, and the solvent is removed in vacuo The crude material is purified by flash chromatograph (SiO₂, DCM-EtOAc 0-10%), and reflashed (SiO₂, hex-EtOAc 0-30%) to afford the title compound as a yellow oil.

¹H NMR (400 MHz, DMSO) δ 6.20 (m, 2H), 5.99 (dd, 1H), 5.63 (d, 1H), 4.18 (m, 1H), 3.60 (s, 3H), 3.17-3.22 (m, 6H), 2.77 (dd, 2H), 2.62 (dd, 2H), 1.79 (m, 2H); MS-APCI (m/z+) 347.1 (M+H).

VI. Preparation of 3-[3,5-difluoro4-(1,4-thiazepanyl)-phenyl]-2-oxo-oxazolidine-5-carboxylic acid methyl ester.

A solution of 3-[3,5-difluoro-4-(1,4-thiazepany)-phenylamino]-2-hydroxypropionic acid methyl ester 6 (3.9 g, 11 mmol) in anhydrous acetonitrile (17 mL) is treated with CDI (2.7 g, 17 mmol) at 23° C. and stirred for 15 min, the solution is then heated to 50 ° C. for 15 h.The reaction mixture is quenched by the addition of H₂O (20 mL) and extracted with EtOAc (3×20 mL). The combined organic layers are washed with brine (50 mL), dried over MgSO4, and the solvent is removed in vacuo The crude material is purified by flash chromatograph (SiO₂, DCM-EtOAc 0-5%), to afford the title compound.

¹H NMR (400 MHz, CDCl₃) δ 7.28 (m, 2H), 5.36 (dd, 1H), 4.32 (dd, 1H), 4.14 (dd, 1H), 3.77 (s, 3H), 3.38 (m, 4H), 2.80 (m, 2H), 2.77 (m, 2H), 1.84 (m, 2H); MS-APCI (m/z+) 373 (M+H).

### Example 2

### Preparation of 3-[3,5-difluoro-4-(1,4-thiazepan-4-yl)-phenyl]-2-oxo-oxazolidine-5-carboxylic acid methylamide

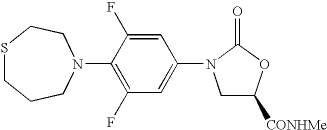

A solution of 3-[3,5-difluoro-4-(1,4-thiazepanyl)-phenyl]-2-oxo-oxazolidine-5-carboxylic acid methyl ester (0.1 g, 0.27 nmnol) in MeOH (1.5 mL) is treated with a methylamine hydrochloride (0.073 g, 1.1 mmol) and then triethylamine (0.18 mL, 1.3 mmol) at room temperature with stirring for 17 h. The solvent is removed under reduced pressure and the crude material is dissolved in DCM/MeOH (10:1) and purified by preparative chromatograph (SiO₂, DCM-MeOH 5%), to afford the title compound as a yellow solid.

¹H NMR (400 MHz, CDCl₃) δ 7.10 (m, 2H), 6.61 (s, 1H), 4.98 (dd, 1H), 4.20 (m, 2H), 3.48 (m, 4H), 2.92 (d, 3H), 2.90 (m, 2H), 2.82 (dd, 2H), 1.90 (m, 2H); MS-APCI (m/z+) 372 (M+H).

### Example 3

### Preparation of 3-[4-(1,1-dioxo-1λ⁶-[1,4]thiazepan-4-yl)-3,5-difluoro-phenyl]-2-oxo-oxazolidine-5-carboxylic acid amide

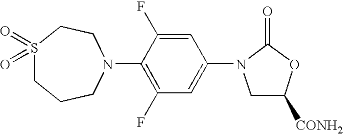

A 5 mL flask charged with 3-[4-(1,1-dioxo-1λ⁶-[1,4]thiazepan-4-yl)-3,5-difluoro-phenyl]-2-oxo-oxazolidine-5-carboxylic acid methyl ester (0.12 g, 0.29 mmol) is treated with a methanolic solution of ammonia (1.5 mL, 2M in MeOH, 3.0 mmol) at room temperature with stirring for 17 h. The solvent is removed under reduced pressure and the crude material is dissolved in DCM/MeOH (10:1) and purified by preparative chromatograph (SiO₂, DCM-MeOH 7%), to afford the title compound as a yellow solid. ¹H NMR (400 MHz, DMSO) δ 7.86 (s, 1H), 7.62 (s, 1H), 7.36 (m, 2H), 5.21 (dd, 1H), 4.23 (dd, 1H), 3.91 (dd, 1H), 3.43 (m, 2H), 3.27 (m, 6H), 2.07 (m, 2H); MS-APCI (m/z+) 390 (M+H).

Intermediate for the synthesis of 3 is prepared as follows.

I. Preparation of 3-[4-(1,1-dioxo-1λ⁶-[1,4]thiazepan-4-yl)-3,5-difluoro-phenyl]-2-oxo-oxazolidine-5-carboxylic acid methyl ester

A solution of 3-[3,5-difluoro-4-(1,4)-thiazepanyl-phenyl]-2-oxo-oxazolidine-5-carboxylic acid methyl ester (0.5 g, 1.3 mmol) in DCM (13 mL) is treated with m-CPBA (0.93 g, 5.4 mmol) at room temperature and stirred for 2 h under N₂. The reaction is quenched with saturated NaHCO₃ (10 mL), the layers are separated and the aqueous layer is extracted into CH₂Cl₂ (3×10 mL). The combined organic layers are washed with brine 20 mL), dried over MgSO₄, filtered, and then concentrated under reduced pressure. The crude material is purified by flash chromatograph (SiO₂, DCM-EtOAc 0-10%), to afford the title compound as a yellow solid.

¹H NMR (400 MHz, DSMO) δ 7.27 (m, 2H), 5.32 (dd, 1H), 4.25 (dd, 1H), 4.15 (dd, 1H), 3.72 (s, 3H), 3.40 (m, 2H), 3.21 (m, 6H), 2.05 (m, 2H); MS-APCI (m/z+) 405.1 (M+H).

### Example 4

### Preparation of 3-[4-(1,1-dioxo-1λ⁶-[1,4]thiazepan-4-yl)-3,5-difluoro-phenyl]-2-oxo-oxazolidine-5-carboxylic acid methylamide

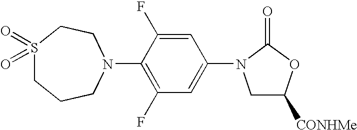

A solution of 3-[4-(1,1-dioxo-1λ⁶-[1,4]thiazepan-4-yl)-3,5-difluoro-phenyl]-2-oxo-oxazolidine-5-carboxylic acid methyl ester (0.12 g, 0.28 mmol) in MeOH (1.5 mL) is treated with methylamine hydrochloride (0.077 g, 1.2 mmol) and then triethylamine (0.19 mL, 1.4 mmol) at room temperature with stirring for 17 h. The solvent is removed under reduced pressure and the crude material is dissolved in DCM/MeOH (10:1) and purified by preparative chromatograph (SiO₂, DCM-MeOH 7%), to afford the title compound as a yellow solid.

¹H NMR (400 MHz, DMSO) δ 8.37 (m, 1H), 7.34 (m, 2H), 5.06 (dd, 1H), 4.24 (dd, 1H), 3.99 (m, 1H), 3.42 (m, 2H), 3.27 (m, 6H), 2.65 (d, 3H), 2.07 (m, 2H); MS-APCI (m/z+) 404.1 (M+H).

### Example 5

### Preparation of 3-[4-(1,1-dioxo-1λ⁶-[1,4]thiazepan-4-yl)-3,5-difluoro-phenyl]-2-oxo-oxazolidine-5-carboxylic acid methoxy-amide

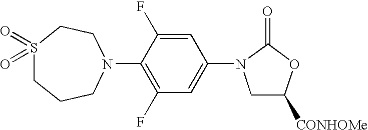

A solution of 3-[4-(1,1-dioxo-1λ⁶-[1,4]thiazepan-4-yl)-3,5-difluoro-phenyl]-2-oxo-oxazolidine-5-carboxylic acid methyl ester (0.13 g, 0.32 mmol) in MeOH (1.5 mL) is treated with methoxylamine hydrochloride (0.087 g, 1.3 mmol) and then triethylamine (0.22 mL, 1.6 mmol) at room temperature with stirring for 17 h. The solvent is removed under reduced pressure and the crude material is dissolved in DCM/MeOH (10:1) and purified by preparative chromatograph (SiO₂, DCM-MeOH 5%), to afford the title compound as a yellow solid.

¹H NMR (400 MHz, DMSO) δ 11.80 (s, 1H), 7.35 (m, 2H), 5.01 (dd, 1H), 4.22 (dd, 1H), 4.05 (m, 1H), 3.63 (s, 3H), 3.43 (m, 2H), 3.27 (m, 6H), 2.07 (m, 2H); MS-APCI (m/z+) 420.1 (M+H).

### Example 6

### Preparation of 3-{3,5-difluoro-4-[1-(2,2,2-trifluoro-acetylimino)-114-[1,4]thiazepan-4-yl]-phenyl}-2-oxo-oxazolidine-5-carboxylic acid amide

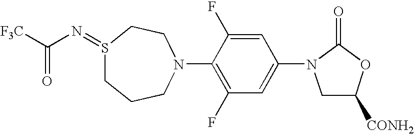

A 5 mL flask charged with 3-{3,5-difluoro4-[1-(2,2,2-trifluoro-acetylimino)-114-[1,4]thiazepan-4-yl]-phenyl}-2-oxo-oxazolidine-5-carboxylic acid methyl ester (0.065 g, 0.13 mmol) is treated with a methanolic solution of ammonia (0.67 mL, 2M in MeOH, 1.3 mmol) at room temperature with stirring for 17 h. The solvent is removed under reduced pressure and the crude material is dissolved in DCM/MeOH (10:1) and purified by preparative chromatograph (SiO₂, DCM-MeOH 7%), to afford the title compound as yellow oil.

¹H NMR (400 MHz, CD₃OD) δ 7.32 (m, 2H), 5.07 (dd, 1H), 4.31 (dd, 1H), 4.08 (dd, 1H), 3.73 (m, 4H), 3.57 (m, 1H), 3.41 (m, 2H), 3.24 (m, 1H), 2.31 (m, 1H), 2.19 (m, 1H); MS-APCI (m/z+) 469.1 (M+H).

The intermediate for the synthesis of Example 6 is prepared as follows:
I. 3-{3,5-difluoro-4-[1-(2,2,2-trifluoro-acetylimino)-1λ⁴-[1,4]thiazepan-4-yl]-phenyl}-2-oxo-oxazolidine-5-carboxylic acid methyl ester
(Ref Okamura H.; Bohm, C. Org. Lett. 2004, 6, 1305-1307.) A suspension of 3-[3,5-difluoro-4-(1,4)-thiazepanyl-phenyl]-2-oxo-oxazolidine-5-carboxylic acid methyl ester (0.2 g, 0.54 mmol) in DCM (6 mL) is treated with trifluoroacetamide (0.12 g, 1.1 mmol), MgO (0.087 g, 2.1 mmol), Rh₂(Oac)₄ (0.005 g, 0.013 mmol) and PhI(Oac)₂ (0.26 g, 0.81 mmol) at room temperature with stirring for 4 h. The reaction mixture is passed through a short plug of silica gel, washed with DCM-MeOH 5% (50 mL) and the solvent is removed in vacuo. The crude material is purified by flash chromatograph (SiO₂, hexane-EtOAc 10-50%), to afford the title compound as a yellow oil.

¹H NMR (400 MHz, DSMO) δ 7.17 (m, 2H), 5.02 (dd, 1H), 4.20 (dd, 1H), 4.02 (dd, 1H), 3.82 (s, 3H), 3.71 (m, 1H), 3.60 (m, 1H), 3.57 (m, 1H), 3.38 (m, 4H), 2.37 (m, 1H), 2.17 (m, 1H); MS-APCI (m/z+) 484.1 (M+H).

### Example 7

### Preparation of 3-{3,5-difluoro-4-[1-(2,2,2-trifluoro-acetylimino)-1λ⁴-[1,4]thiazepan-4-yl]-phenyl}-2-oxo-oxazolidine-5-carboxylic acid methylamide

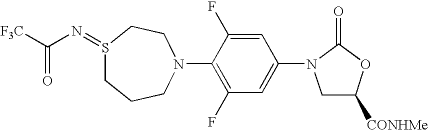

A solution of 3-{3,5-difluoro-4-[1-(2,2,2-trifluoro-acetylimino)-1 4-[1,4]thiazepan-4-yl]-phenyl}-2-oxo-oxazolidine-5-carboxylic acid methyl ester (0.042 g, 0.086 mmol) in MeOH (0.5 mL) is treated with methylamine hydrochloride (0.023 g, 0.35 mmol) and then triethylamine (0.06 mL, 0.43 mmol) at room temperature with stirring for 17 h. The solvent is removed under reduced pressure and the crude material is dissolved in DCM/MeOH (10:1) and purified by preparative chromatograph (SiO₂, DCM-MeOH 7%), to afford the title compound as yellow oil.

¹H NMR (400 MHz, CD₃OD) δ 7.32 (m, 2H), 5.07 (dd, 1H), 4.30 (dd, 1H), 4.06 (dd, 1H), 3.74 (m, 4H), 3.41 (m, 2H), 3.23 (m, 2H), 2.81 (s, 3H), 2.29 (m, 1H), 2.19 (m, 1H); MS-APCI (m/z+) 483.1 (M+H).

### Example 8

### Preparation of (5R)-3-[3-fluoro-4-(1,4-thiazepan-4-yl)-phenyl]-2-oxo-oxazolidine-5-carboxylic acid amide.

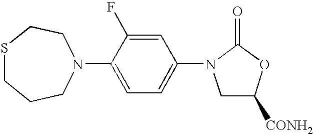

A 50 mL flask charged with 5-(R)-3-[3-fluoro-4-(1,4-thiazepanyl)-phenyl]-2-oxo-oxazolidine-5-carboxylic acid methyl ester (0.3 g, 0.86 mmol) is treated with a methanolic solution of ammonia (4 mL, 2M in MeOH, 8.6 mmol) at room temperature with stirring overnight. The solvent is removed from the tan slurry under reduced pressure. The crude material is dissolved in DCM and purified by preparative chromatograph (SiO₂, DCM-MeOH 5%), to afford the title compound as a white solid.

¹H NMR(400 MHz, DMSO) 7.77 (s, 1H), 7.53 (s, 1H), 7.37 (dd, 1H), 7.11 (m, 1H), 6.94 (dd, 1H),4.95 (dd, 1H), 4.16 (t, 1H), 3.91 (dd, 1H), 3.52 (m, 4H), 2.80 (m, 2H), 2.55 (t, 2H), 1.90 (m, 2H); MS-APCI 10V (m/z+) 340.1 (M+H).

Intermediates for the synthesis of Example 8 are prepared as follows:
I. Preparation of 4-(2-fluoro-4-nitro-phenyl)-1,4-thiazepane

A solution of 3,4-difluoro-nitro-benzene (8.6 g, 54 mmol) in anhydrous acetonitrile (56 mL) is treated with triethylamine (8.3 mL, 59.5 mmol), followed by slow addition of 1,4-thiazepane (7.0 g, 59.5 mmol) suspended in acetonitrile (10 mL) over 20 min. The mixture is stirred at room temperature overnight, then quenched by the addition of H₂O (100 mL) producing a yellow precipitate. The suspension is cooled in an ice bath then the yellow solid is collected and dried in vacuo to give 4-(2-fluoro-4-nitro-phenyl)-1,4-thiazepane (11.1 g, 80%): ¹H NMR (400 MHz, DMSO) δ 7.87 (m, 2H), 6.98 (m, 1H), 3.78 (m, 4H), 2.83 (t, 2H), 2.54 (t, 2H),1.93 (m, 2H) MS-APCI10V (m/z+) 257.0 (M+H).

II. Preparation of 3-fluoro-4-(1,4-thiazepanyl)-phenylamine

A solution of 4-(2-difluoro-4-nitro-phenyl)-1,4-thiazepane (10.5 g, 41 mmol) in absolute ethanol (67 mL) is treated with SnCl₂.2H₂O (37 g, 164 mmol) and refluxed for 1.5 h. The miuture is allowed to cool to room temperature and ice water is added (100 ml), followed by the addition of 50% aqueous NaOH to pH 11. The solution is stirred for 1 h at room temperature and then extracted with CH₂Cl₂ (3×150 mL). The combined organic layers were washed with brine (200 mL), dried over MgSO4, and the solvent is removed in vacuo The crude material is used without further purification to afford the title compound as an orange solid: ¹H NMR (400 MHz, DMSO) δ 6.71 (m, 1H), 6.24 (m, 2H), 4.78 (s, 2H), 3.29 (m, 2H), 3.25 (m, 2H), 2.73 (m, 2H), 2.62 (t, 2H), 1.84 (m, 2H); MS-APCI10V (m/z+) 227.1 (M+H).

III. Preparation of 2(R)-3-[3-fluoro-4-(1,4-thiazepany)-phenylamino]-2-hydroxypropionic acid methyl ester

A solution of 3-fluoro-4-(1,4-thiazepanyl)-phenylamnine (8.5 g, 37.5 mmol) in anhydrous acetonitrile (21 mL) is treated with LiOTf (5.9 g, 37.6 mmol) and (R)-methyl glycidate (3.8 g, 37.6 mmol) and refluxed for 15 h. After 15 h, the reaction is incomplete therefore additional (R)-methyl glycidate (0.40 g, 3.9 mmol) is added and the solution is refluxed for another 24 h. The reaction is still incomplete therefore additional (R)-methyl glycidate (0.40 g, 3.9 mmol) is added and the solution is refluxed for another 8 h. The mixture is allowed to cool to room temperature and quenched by the addition of H₂O (21 mL), then extracted with EtOAc (3×50 mL). The combined organic layers were washed with brine (100 mL), dried over MgSO4, and the solvent is removed in vacuo The crude material (14.35 g) is purified by flash chromatograph (SiO₂, DCM-EtOAc 0-5%), to afford the title compound as a yellow oil: ¹H NMR (400 MHz, DMSO) δ6.67 (m, 1H), 6.36 (dd, 1H), 5.28 (dd, 1H), 5.61 (d, 1H), 5.32 (t, 1H), 4.15 (m, 1H), 3.57 (s, 3H), 3.19-3.33 (m, 4H), 3.11 (m, 1H), 2.74 (m, 2H), 2.61 (t, 2H), 1.85 (m, 2H); MS-APCI10V (m/z+) 329.1(M+H).

IV. Preparation of 2(R)-3-[3-fluoro-4-(1,4-thiazepanyl)-phenyl]-2-oxo-oxazolidine-5-carboxylic acid methyl ester

A solution of 2(R)-3-[3-fluoro-4-(1,4-thiazepany)-phenylamino]-2-hydroxypropionic acid methyl ester (1.8 g, 5.6 mmol) in anhydrous acetonitrile (13 mL) is treated with CDI (1.6 g, 8.4 mmol) at 23° C. and stirred for 15 min, the solution is then heated to 50° C. and held overnight. The reaction mixture is allowed to cool to room temperature before it is quenched by the addition of H₂O (12 mL) and extracted with EtOAc (3×13 mL). The comnbined organic layers were washed with brine (13 mL), dried over MgSO4, and the solvent is removed in vacuo The crude material is purified by flash chromatograph (SiO₂, DCM-EtOAc 0-3%), to afford the title compound as a beige solid: ¹H NMR (400 MHz, DMSO) δ7.29 (dd, 1H), 7.08 (m, 1H), 6.89 (dd, 1H), 5.22 (dd, 1H), 4.25 (t, 1H), 4.03 (dd, 1H), 3.70 (s, 3H), 3.52 (m, 4H), 2.80 (m, 2H), 2.54 (t, 2H), 1.89 (m, 2H); MS-APCI 10V (m/z+) 355.1 (M+H).

### Example 9

### 5(R)-3-[3-Fluoro-4-(1,4-thiazepan-4-yl)-phenyl]-2-oxo-oxazolidine-5-carboxylic acid methylamide

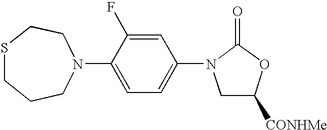

A solution of 5-(R)-3-[3-fluoro-4-(1,4-thiazepanyl)-phenyl]-2-oxo-oxazolidine-5-carboxylic acid methyl ester (0.32 g, 0.90 mmol) in MeOH (4 mL) is treated with a methylamine hydrochloride (0.24 g, 3.6 mmol) and then triethylarnine (0.63 mL, 4.5 mmol) at room temperature with stirring overnight. The resulting white suspension is filtered and wetcake is rinsed twice with 2 ml of MeOH to afford the title compound as a white solid.

¹H NMR (400 MHz, DMSO) 8.30(s, 1H), 7.36 (dd, 1H), 7.10 (dd, 1H), 6.91 (t, 1H), 4.99 (dd, 1H), 4.17 (t, 1H), 3.90 (dd, 1H), 3.53 (m, 4H), 2.80 (m, 2H), 2.61 (d, 3H), 2.55 (m, 2H), 1.90 (m, 2H); MS-APCI 10V (m/z+) 354.1 (M+H).

### Example 10

### 5(R)-3-[4-(1-oxo-λ⁶-[1,4]thiazepan-4-yl)-3-fluoro-phenyl]-2-oxo-oxazolidine-5-carboxylic acid methyl ester

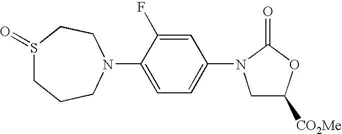

A solution of 5(R)-3-[3-fluoro-4-(1,4)-thiazepanyl-phenyl]-2-oxo-oxazolidine-5-carboxylic acid methyl ester (0.5 g, 1.6 mmol) in 50/50 DCM/MeOH (30 mL) cooled to 0° C. is treated with periodate (0.40 g, 1.9 mmol) and allowed to warm to room temperature overnight with stirring under N₂. The reaction is quenched with water (13 mL), then extracted 3×'s with 13 ml of DCM. The combined organic layers were washed with brine (25 mL), dried over MgSO₄, filtered, and then concentrated under reduced pressure to give 0.55 g of a light brown solid. The crude material is purified by flash chromatograph (SiO₂, DCM-EtOAc 0-5%), to afford the title comopund as a yellow solid.

¹H NMR (400 MHz, DSMO) 7.41 (dd, 1H), 7.14 (dd, 1H), 6.98 (dd, 1H), 5.27 (dd, 1H), 4.29 (td, 1H), 4.08 (m, 1H), 3.70 (s, 3H), 3.53-3.60 (m, 1H), 3.30-3.39 (m, 2H), 3.14 (m, 1H), 2.96 (m, 3H) 2.81-2.87 (m, 11H), 2.33(m, 1H), 1.89(m, 1H), MS-APCI 10V (m/z+) 371.1(M+H).

### Example 11

### 5(R)-3-[-fluoro-4-(1-oxo-1λ⁴-[1,4]thiazepan-4yl)-phenyl]-2-oxo-oxazolidine-5-carboxylic acid methyl amide

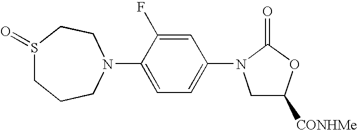

A solution of 5(R)-3-[4-(1-oxo-λ⁶--[1,4]thiazepan-4-yl)-3-fluoro-phenyl]-2-oxo-oxazolidine-5-carboxylic acid methyl ester ( 0.12 g 0.33 mmol) in MeOH (2 ml) is treated with methylamine hydrochloride (0.09 g 1.3 mmol) followed by triethylamine (0.17 g 1.7 mmol) at room temperature with stirring overnight. The resulting white suspension is filtered and wetcake is rinsed with MeOH to afford the compound a white solid.

¹H NMR (400 MHz, DSMO) 8.31(s, 1H), 7.43 (dd, 1H), 7.14 (dd, 1H), 6.98 (t, 1H), 5.00(dd, 1H), 4.18 (t, 1H), 3.91 (m, 1H), 3.59 (m, 1H), 3.38 (m, 2H), 3.16(m, 1H), 2.82-3.04 (m, 4H), 2.61 (d, 3H), 2.33 (m, 1H), 1.90 (m, 1H); MS-APCI 10V (m/z+) 370.1 (M+H).

### Example 12

### 5(R)-3-[3-fluoro4-(1-oxo-1-[1,4]thiazepan-4-yl)-phenyl]-2-oxo-oxazolidine-5-carboxylic acid amide

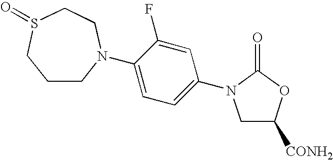

A solution of 5(R)-3-[4-(1-oxo-λ⁶-[1,4]thiazepan-4-yl)-3-fluoro-phenyl]-2-oxo-oxazolidine-5-carboxylic acid methyl ester (0.11 g, 0.30 mmol) is treated with a methanolic solution of ammonia (2 mL, 2M in MeOH, 3.0 mmol) at room temperature with stirring overnight. The resulting tan suspension is filtered leaving a white solid, which is rinsed with MeOH and dried to afford the title compound as a white solid.

¹H NMR(400 MHz, DMSO) 7.39 (dt 1H), 7.11 (dt, 1H), 6.95 (dd, 1H), 4.93 (dd, 1H), 4.16 (td, 1H), 3.88 (m, 1H), 3.56 (m, 1H), 3.52 (s, 2H), 3.36 (mn, 1H), 3.26 (m, 1H), 3.13 (m, 1H), 3.00 (m, 3H), 2.89 (m, 1H), 2.31 (m, 1H), 1.90 (m, 1H). MS-APCI 10V (m/z+) 356.1 (M+H).

### Example 13

### Preparation of 5(R)-3-[4-(1,1-dioxo-1λ⁶-[1,4]thiazepan-4-yl)-3-fluoro-phenyl]-2-oxo-oxazolidine-5-carboxylic acid methyl amide

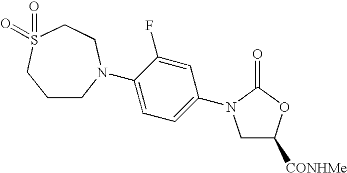

3-[4-(1,1-Dioxo-1λ⁶-[1,4]thiazepan-4-yl)-3-fluoro-phenyl]-2-oxo-oxazolidine-5-carboxylic acid methyl ester (0.08 g 0.20 mmol) suspended in MeOH (10 ml) was treated with methylamine hydrochloride (0.06 g 0.82 mmol) followed by triethylamine (0.10 g 1.0 mmol) at room temperature and held overnight with stirring. The resulting white suspension was filtered and the wetcake rinsed with MeOH to afford 3-[4-(1,1-Dioxo-1λ⁶-[1,4]thiazepan-4-yl)-3-fluoro-phenyl]-2-oxo-oxazolidine-5-carboxylic acid methyl amide (0.04 g, 51%) as a white solid. 1H NMR (400 MHz, DSMO) 8.31 (d, 1H), 7.44 (dd, 1H), 7.16 (m, 1H), 7.01 (dd, 1H), 5.00(dd, 1H), 4.18 (t, 1H), 3.93 (dd, 1H), 3.49 (m, 2H), 3.37 (m, 4H), 3.20(m, 2H), 2.61 (d, 3H), 2.06 (m, 2H); MS-APCI 10V (m/z+) 386.0 (M+H).

This compound was prepared from the following intermediate:

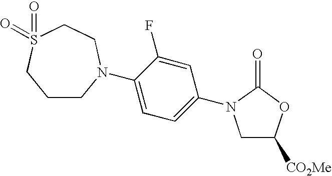

I. Preparation of 3-[4-(1,1-Dioxo-1λ⁶-[1,4]thiazepan-4-yl)-3-fluoro-phenyl]-2-oxo-oxazolidine-5-carboxylic acid methyl ester.

A solution of 3-[4-(1-oxo-λ⁶-[1,4]thiazepan-4-yl)-3-fluoro-phenyl]-2-oxo-oxazolidine-5-carboxylic acid methyl ester (0.50 1.41 mmol) in Dichloromethane (14 ml) was treated with 3-chloroperoxybenzoic acid (1.27 g 5.6 mmol) at room temperature with stirring overnight. The reaction was quenched with 10 ml of saturated aqueous NaHCO₃ phases were separated and the aqueous was extracted 3 additional times with dichloromethane (5 ml). The combined organics were washed with brine (5 ml), dried over MgSO4 then concentrated under vacuum to yield a yellow solid. The crude material was purified using preparative thin layer chromatography (silica with 10% MeOH in Dichloromethane) to afford 3-[4-(1,1-Dioxo-1λ⁶-[1,4]thiazepan-4-yl)-3-fluoro-phenyl]-2-oxo-oxazolidine-5-carboxylic acid methyl ester (0.08 g, 14.5%) as a white solid.

¹H NMR (400 MHz, DSMO) 7.41(dd, 1H), 7.16 (dd, 1H), 7.02 (dd, IH), 5.27 (dd, 1H), 4.26(t, 1H), 4.08 (dd, 1H), 3.70 (s, 3H), 3.49 (n, 2H), 3.38 (in, 4H), 3.20(m, 2H), 2.06 (m, 2H); MS-APCI 10V (m/z+) 387.0 (M+H).

We claim:

1. A compound of formula I 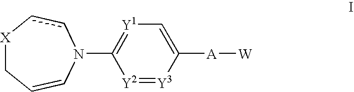
or a pharmaceutically acceptable salt thereof wherein:
A is a structure of the following formula i, ii, iii, or iv 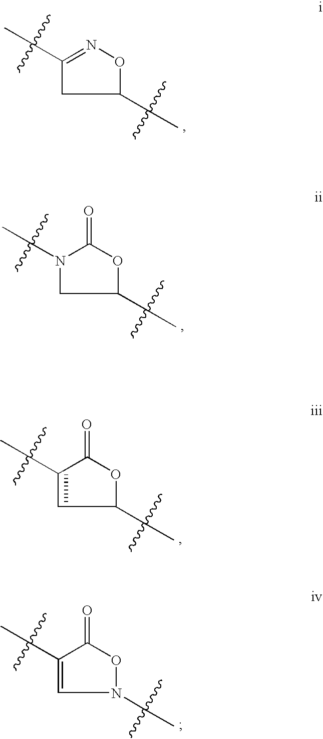
W is
(a) CONHR¹,
(b) CH₂NHR²,
(c) CH₂OH,
(d) CH(OH)—CH=CHR¹,
(e) CH(OH)C≡CR¹,
(f) CH₂NH-het,
(g) CH₂O-het,
(h) CH₂S-het, or
(i) CH₂het;
X is S, SO, SO₂, or S=N-C(=O)C₁₋₆alkyl;
Y¹ is CH, CF, or N;
Y² and Y³ are independently CH or CF;
R¹ is H, C₁₋₆ alkyl, or OC₁₋₆alkyl;
R² is CO₂(NH)C₁₋₄alkyl;
each “. . . ” is independently a bond or absence;
at each occurrence, C₁₋₆alkyl is optionally substituted with one or more CF₃, halo, OH, OC₁₋₄alkyl, CN, N₃, O(C=O)C₁₋₄alkyl, C₃₋₆cycloalkyl, NH₂, NHC(=O)C₁₋₄alkyl, or C(=O)C₁₋₄alkyl; and
het is a five- (5) or six- (6) membered heterocyclic ring having 1-4 heteroatoms selected from the group consisting of oxygen, sulfur, and nitrogen within the ring,
wherein each carbon atom in het is optionally substituted with one or more CF₃, halo, OH, OC₁₋₄alkyl, CN, N₃, O(C=O)C₁₋₄alkyl, C₃₋₆cycloalkyl, NH₂, NH₂, NHC(=O)C₁₋₄alkyl, or C(=O)C₁₋₄ alkyl.

2. A compound of claim 1 which is a compound of formula Ib 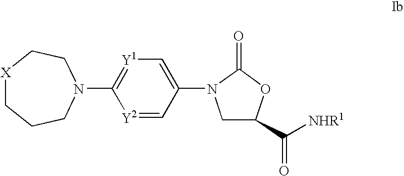
wherein R¹ is H, CH₃ or OCH₃.

3. A compound of claim 2 wherein X is SO₂.

4. A compound of claim 1 which is
(a) 3-[3,5-difluoro4-(1,4-thiazepan-4-yl)-phenyl]-2-oxo-oxazolidine-5-carboxylic acid amide,
(b) 3-[3,5-difluoro-4-(1,4-thiazepan-4-yl)-phenyl]-2-oxo-oxazolidine-5-carboxylic acid methylamide,
(c) 3-[4-(1,1-dioxo-1λ⁶-[1,4]thiazepan-4-yl)-3,5-difluoro-phenyl]-2-oxo-oxazolidine-5-carboxylic acid amide,
(d) 3-[4-(1,1-dioxo-1λ⁶-[1,4]thiazepan-4-yl)-3,5-difluoro-phenyl]-2-oxo-oxazolidine-5-carboxylic acid methylamide,
(e) 3-[4-(1,1-dioxo-1λ⁶-[1,4]thiazepan-4-yl)-3,5-difluoro-phenyl]-2-oxo-oxazolidine-5-carboxylic acid methoxy-amide,
(f) 3-{3,5-difluoro-4-[1-(2,2,2-trifluoro-acetylimino)-114-[1,4]thiazepan-4-yl]-phenyl}-2-oxo-oxazolidine-5-carboxylic acid amide,
(g) 3-{3,5-difluoro4-[1-(2,2,2-trifluoro-acetylimino)-114-[1,4]thiazepan-4-yl]-phenyl}-2-oxo-oxazolidine-5-carboxylic acid methylamide,
(h) (5R)-3-[3-fluoro4-(1,4-thiazepan-4-yl)-phenyl]-2-oxo-oxazolidine-5-carboxylic acid amide,
(i) 5(R)-3-[3-fluoro-4-(1,4-thiazepan-4-yl)-phenyl]-2-oxo-oxazolidine-5-carboxylic acid methylamide,
(j) 5(R)-3-[-fluoro-4-(1-oxo-1λ⁴-[1,4]thiazepan-4yl)-phenyl]-2-oxo-oxazolidine-5-carboxylic acid methyl amide,
(k) 5(R)-3-[3-fluoro-4-(1-oxo-1λ⁴-[1,4]thiazepan-4-yl)-phenyl]-2-oxo-oxazolidine-5-carboxylic acid amide, or
(l) 5(R)-3-[4-(1,1-dioxo-1λ⁶-[1,4]thiazepan-4-yl)-3-fluoro-phenyl]-2-oxo-oxazolidine-5-carboxylic acid methyl amide.

5. A pharmaceutical composition comprising a compound of claim 1 or a pharmaceutically acceptable salt thereof, and a pharmaceutically acceptable carrier.

6. A use of a compound of claim 1 for the preparation of a medicament for treating bacteria infectious diseases

7. The use of claim 6 wherein the compound of claim 1 is administered orally, parenterally, topically, rectally, or intranasally.

8. The use of claim 6 wherein said compound is administered in an amount of from about 0.1 to about 100 mg/kg of body weight/day.

9. The bacteria infectious diseases of claim 6 which is ear infections, eye infections, respiratory tract infections, skin and skin structure infections, bacterial endocarditis, osteomyelitis, endocarditis or diabetic foot.

10. The bacteria infectious diseases of claim 6 which is caused by gram-positive bacteria, gram negative bacteria, anaerobic organisms, and acid-fast organisms.

11. The bacteria infectious diseases of claim 6 which is caused by bacteria comprising staphylococci, streptococci, Enterococci, Haemophilus, Moraxella, bacteroides, clostridia, Mycobacteria, or Chlamydia.

12. The bacteria of claim 11 wherein staphylococci is S. aureus and S. epidermidis; wherein streptococci is S. pneumoniae of S. pyogenes; wherein Enterococci is E. faecalis; wherein Haemophilus is H. influenzae; wherein Moraxella is M. catarrhalis; and wherein Mycobacteria is M. tuberculosis; or Mycobacterium avium.

13. The bacteria infectious diseases of claim 6 which is community-acquired pneumoniae or infections caused by multi-drug resistant S. aureus.
